(19) United States
(12) Patent Application Publication
(10) Pub. No.: US 2005/0280531 A1
(43) Pub. Date: Dec. 22, 2005

(54) Device and method for transmitting physiologic data

(76) Inventors: Kalford C. Fadem, Louisville, KY (US); Benjamin A. Schnitz, Brentwood, TN (US)

Correspondence Address: FROST BROWN TODD, LLC, 2200 PNC CENTER, 201 E. FIFTH STREET, CINCINNATI, OH, 45202, US

(21) Appl. No.: 11/154,192

(22) Filed: Jun. 16, 2005

Related U.S. Application Data
(60) Provisional application No. 60/580,776, filed on Jun. 18, 2004.
(60) Provisional application No. 60/580,772, filed on Jun. 18, 2004.

(51) Int. Cl. G08B 1/08; A61B 5/00; H04Q 7/00

(52) U.S. Cl. 340/539.12; 128/903; 600/549; 600/323; 340/539.13

(57) ABSTRACT

A wireless biopotential monitoring system composed of a wireless electrode module which can be attached to a disposable electrode strip. Such a device can be conveniently affixed to a patient's skin and will transmit the physiological signals to a remote receiver where the signals can be monitored by a clinician. The device is powered by a fuel-air battery. The device would remain packaged in an airtight package until it needs to be applied at which time either the wounded soldier would apply the device himself/herself or it would be applied by another soldier or corpsman. The device would begin to measure brainwave activity, heart rate, and dissolved oxygen level. The device would also identify the wounded soldier's location using the onboard GPS receiver. The physiologic data along with the soldier's position would then be transmitted to a remote receiver.

### REFERENCE TO RELATED APPLICATIONS

The present application claims the benefit of U.S. Patent Appl. Ser. No. 60/580,776, “DEVICE AND METHOD FOR TRANSMITTING PHYSIOLOGIC DATA” and 60/580,772, “WIRELESS ELECTRODE FOR BIOPOTENTIAL MEASUREMENT”, both to Fadem et al. and filed on 18 Jun. 2004, the disclosure of both of which are incorporated by reference in its entirety.

### FIELD THE INVENTION

The present invention relates generally to a method and apparatus for monitoring physiologic activity from a remote location. More specifically, the present invention describes a wireless sensor device which can be used to perform various physiologic monitoring functions such as electroencephalography, electrocardiography, and pulse oximetry from a wounded soldier and transmit that information, along with his/her location on the battlefield, to a remote receiver.

### BACKGROUND OF THE INVENTION

As a soldier becomes wounded on the battlefield, the officers and corpsmen must decide when to put additional troops at risk to retrieve their wounded comrade. This often results in additional casualties even when the initial wounded soldier either does not have a life threatening injury or has already died from his/her wounds. It would be preferable if the officers and corpsmen could know the condition of the wounded soldiers as well as their exact location so they could devise a retrieval plan that would get the quickest possible care to those soldiers who have life threatening injuries without unnecessarily putting additional soldiers at risk.

Consequently, a significant need exists for a device for remotely assessing severity of injury that would be suitable for austere conditions.

### BRIEF SUMMARY OF THE INVENTION

The invention describes a device comprising an adhesive strip to be applied to a location on the soldier's skin such as the forehead upon being wounded on the battlefield. A biopotential measurement device is thereby activated to detect a physiological voltage potential (e.g., EEG, ECG). This being a weak signal, the sensed voltage potential is signal amplified and converted to a digital signal for wireless data transmission. Thereby, an injured or wounded patient may be remotely located and medically assessed while in an austere, inhospitable situation.

In one aspect of the invention, a device has a substrate that is affixable to skin of a subject to position a pair of electrodes to detect a biopotential signal and to position a transducer to detect a physical parameter of the subject. Also integral to the substrate, a power supply powers a communication interface and circuitry that is operatively configured to amplify and digitize the biopotential detected across the pair of electrodes, to access an identifier associated with the subject, to digitize the physical parameter of the subject, and to communicate a patient status on the communication interface. Thereby, the condition of a wounded, ill or injured subject may be monitored remotely until safe or otherwise warranted to locate and treat.

In another aspect of the invention, a device for monitoring the physiological condition of a person has a flexible substrate including an adhesive undersurface positionable on the skin of the person. Applying this substrate positions a biosensor into contact with the skin to sense a physiological condition. An integral battery powers the attached global positioning system (GPS) receiver and circuitry. The latter converts and transmits the sensed physiological signal from the biosensor as a digital signal with a sensed position from the global positioning system receiver. Thereby emergency responders can plan a suitable and expedient retrieval of the subject with knowledge of the current physiological condition and location of the person.

In another aspect of the invention, a device with a substrate affixable to the skin of a subject positions a pair of electrodes to detect the biopotential of the subject. Battery-powered circuitry operates an emergency beacon and a two-way communication interface that includes a spread spectrum transmitter to communicate the medical condition and the location of the subject.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is a view of the battlefield trauma telemetry system installed on a human subject.

FIG. 2 is a disassembled view of the battlefield trauma telemetry system of FIG. 1.

FIG. 3 is a functional block diagram of the battlefield trauma telemetry system of FIG. 1.

### DETAILED DESCRIPTION OF THE INVENTION

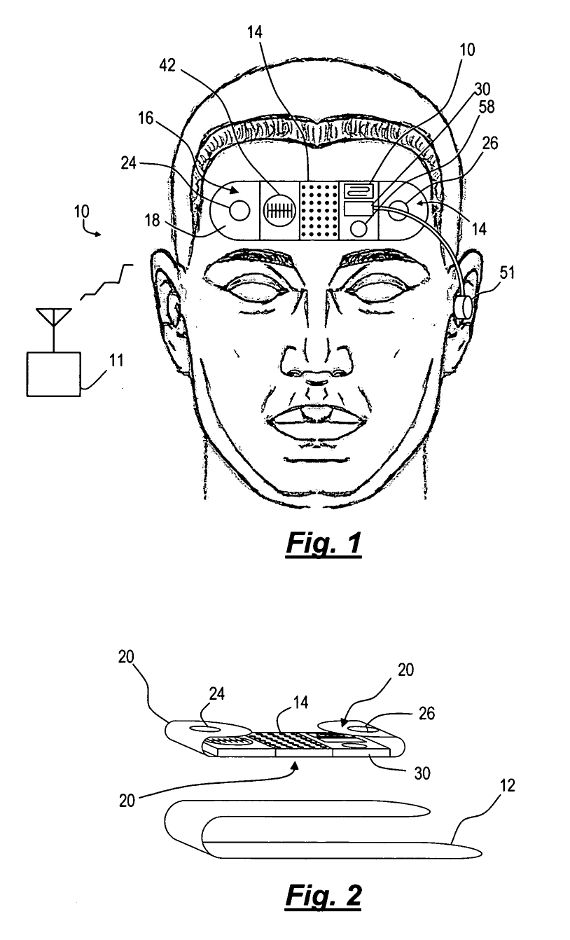

In FIGS. 1-3, a battlefield trauma telemetry system 10 provides a self-contained, disposable apparatus with a long shelf-life that may be readily applied to the skin of a wounded or injured individual. Automated power-up and operation of the battlefield trauma telemetry system 10 thereafter ascertains and communicates the vital health statistics of the wounded or injured individual to a remote transceiver 11 (FIG. 1), which may facilitate search and rescue, triage operations in situations of scarce resources, and/or reduce exposure of individuals to hostile fire in contingency or battlefield conditions.

Packaging such as a peel-off backing 12 (FIG. 2) is readily familiar to the user, avoiding the necessity of any medical or technical training, although printed instructions may enhance proper placement. With the peel-off backing 12 removed, a fuel-air battery 14 (e.g., ZINC-AIR batteries by ELECTRIC FUEL CORPORATION) is activated by the supply of oxygen, thus providing power to an attached integrated adhesive strip 16, although other forms of power may be incorporated. A flexible substrate 18 thereof serves as a flexible printed circuit board (PCB) incorporating conductive traces printed or formed thereon that connect to the fuel-air battery 14 as well as other integrated or discrete electronic components. A waterproof surface or coating (not shown) may prevent precipitation or sweat from sorting out such traces. The flexible substrate 18 has adhesive 20 applied to a bottom surface 22.

Signal and reference electrodes 24, 26 are spatially separated and exposed on the bottom surface 22 to make conductive contact with the skin to detect a biopotential signal (e.g., Electroencephalogram/Electrocardiogram EEG/ECG electrodes as in BIS™ electrodes by ASPECT MEDICAL SYSTEMS). The electrodes 24, 26 may advantageously be part of active EEG circuitry 28 that incorporate active signal processing and amplification as described in the co-pending U.S. patent application Ser. No. 11/092,395, the disclosure of which is hereby incorporated by reference in its entirety. Thereby, biopotentials that may be sensed with varying magnitudes giving variability in placement, skin conductivity, etc., are automatically configured for telemetry.

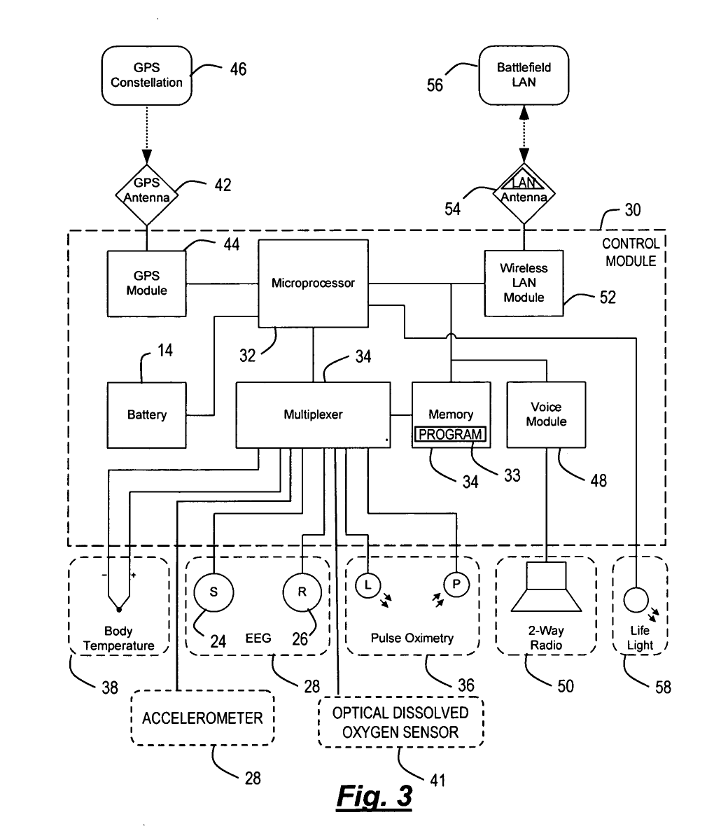

With particular reference to FIG. 3, in the illustrative version, a control module 30, mounted on the flexible adhesive strip 16, contains a microprocessor 32 that executes a program 33 contained in a memory 34. With power applied, the processing may include an initial non-emitting state wherein the visual, audio and/or electromagnetic emissions are disabled until locally or remotely activated. This feature may be particularly advantageous for military use in which detection by hostile forces is undesirable. This feature may also be useful to extend the life of the system 10 by intermittently providing bio status information.

The microprocessor 32 may access and/or control via a multiplexer 34 the electrode 24, 26 as well as other sensors. For instance, a pulse oximetry sensor 36, as in MAX-FAST™ forehead sensor by NELLCOR®, monitors pulse rate. Other sensors may include a temperature sensor 38 (e.g., thermister or thermocouple) for detecting an onset of hypothermia or shock. An integral motion detector, such as an accelerometer 40, may advantageously detect pulse, breathing and/or bodily movements of the wearer. An optical dissolved oxygen sensor 41 may illuminate the skin and measure the wounded or injured individual's breathing difficulty. Although not depicted, some analog sensors may be used with an analog-to-digital converter (not shown).

In addition to bio status information, ambient or environmental conditions may be advantageously sensed, such as position. To that end, the control module 30 incorporates a global positioning system (GPS) antenna 42 and GPS receiver 44, such as in LASSEN™ SQ GPS module by TRIMBLE®, which accurately identifies the location of the battlefield trauma telemetry system 10 with reference to a GPS satellite constellation 46. Unique identification of the wounded or injured individual and/or the battlefield trauma telemetry system 10 may be hardcoded or set by the user so that control information and/or telemetry data may be uniquely associated with the particular system 10.

In addition to data telemetry, emergency two-way radio capabilities may be provided by an audio codex (voice module) 48 controlled by the microcontroller 32 that generates and receives audio via a speaker 50. In FIG. 1, the speaker 50 is depicted as an earphone 51 for output audio with a microphone (not shown) integral with adhesive strip 16. This digitized audio information is then transceived by a wireless local area network (LAN) radio module 52 such as in CH SERIES RF TRANSMITTER by LINX TECHNOLOGIES CORPORATION attached to a LAN antenna 54, which in turn communicates a battlefield LAN 56. For instance, a voice-activated radio may advantageously allow hands off use, as well as provide a means for rescuers to listen in on the locale of the patient prior to extrication. Alternatively or in addition, an interface (not shown) may be included for connecting to a two-way radio carried by the patient or caregiver, thereby taking advantage of its increased range and connectivity. Another function provided by the LAN radio module 52 and LAN antenna 54 may include serving as a one-way emergency locator beacon that is detected by satellite on one or more of frequencies 121.5, 243.0 and 406 MHz, similar to that included in survival kits and parachutes.

To further reduce susceptibility to detection, modulating with a spread spectrum carrier is difficult for hostile forces to differentiate from background noise and provides an additional layer of encryption even if detected. For instance, the newly approved federal Advanced Encryption Standard (AES) endorsed by the National Institute of Standards and Technology (NIST) may be employed, similar to 128-bit AES AIRFORTRESS WIRELESS SECURITY GATEWAY developed by Fortress Technologies Inc. of Tampa, Fla. This device encrypts everything from the data layer up in a wireless local area network (LAN), including holes routinely exploited by hackers, such as IP addresses.

The control module 30 may control a life light, such as an organic light-emitting diode (OLED), attached to an upper surface 60 of the integrated adhesive in the visible or infrared spectrum to help the corpsmen locate the wounded soldier. The light frequency may advantageously be selected for being visible by night vision goggles (NVG), low light camera and/or naked eye. The light pattern/color may also relay information as to the wounded or injured person's condition such as flashing if in a critical condition.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

1. A device, comprising:
a substrate affixable to the skin of a subject;
a pair of electrodes spaced upon an inner surface of the substrate to detect a biopotential of the subject;
a transducer attached to the inner surface of the substrate to detect a physical parameter of the subject;
a power supply attached to the substrate;
a communication interface; and
circuitry operatively configured to amplify and digitize the biopotential detected across the pair of electrodes, to access an identifier associated with the subject, to digitize the physical parameter of the subject, and to communicate a patient status on the communication interface.

2. The device of claim 1, wherein the identifier associated with the subject comprises a geographical location, the device further comprising a positioning sensing device.

3. The device of claim 2, wherein the positioning sensing device comprises a global positioning system receiver.

4. The device of claim 1, wherein the transducer comprises a temperature sensor.

5. The device of claim 1, wherein the transducer comprises a dissolved oxygen sensor.

6. The device of claim 1, wherein the transducer comprises a motion sensor.

7. The device of claim 6, wherein the motion sensor comprises an accelerometer.

8. The device of claim 1, wherein the transducer comprises a pulse oximeter.

9. The device of claim 1, wherein the communication interface comprises a light.

10. The device of claim 9, wherein the circuitry is further operably configured to modulate the light to indicate the patient condition.

11. The device of claim 1, wherein the communication interface comprises a radio transmitter.

12. The device of claim 1, wherein the communication interface comprises a two-way radio transceiver.

13. The device of claim 12, wherein the circuitry is further operably configured to communicate the patient status over the two-way radio transceiver in response to an authenticated received command from the two-way radio transceiver.

14. The device of claim 12, wherein the two-way radio transceiver comprises a spread spectrum local access network (LAN) transceiver.

15. The device of claim 12, further comprising an audio input and audio output device attached to the substrate

16. The device of claim 1, wherein the substrate comprises a flexible substrate having an adhesively coated undersurface.

17. The device of claim 1, wherein the power supply comprises a fuel-air battery.

18. The device of claim 1, wherein the substrate comprises a flexible substrate having an adhesively coated undersurface, the device further comprising a peel-off backing positioned to form a barrier between the fuel-air battery and activating air.

19. The device of claim 1, wherein the circuitry further comprises a memory, a program contained in the memory, and a microprocessor operably configured to execute the program.

20. The device of claim 1, wherein the identifier associated with the subject comprises a unique serial number assigned to the device.

21. The device of claim 1, wherein the identifier associated with the subject comprises a unique serial number assigned to the subject, the device further comprising an input port operably configured to receive the unique serial number.

22. A device for monitoring the physiological condition of a person, comprising:
a flexible substrate including an adhesive undersurface positionable on the skin of the person;
a biosensor attached to the undersurface of the flexible substrate;
a battery attached to the flexible substrate;
a global positioning system receiver attached to the flexible substrate; and
circuitry powered by the battery and attached to the flexible substrate and operably configured to convert and transmit a sensed physiological signal from the biosensor as a digital signal with a sensed position from the global positioning system receiver.

23. A device, comprising:
a substrate affixable to the skin of a subject;
a pair of electrodes spaced upon on an inner surface of the substrate to detect a biopotential of the subject;
a battery attached to the substrate;
an emergency beacon;
a two-way communication interface including a spread spectrum transmitter; and
circuitry operatively configured to amplify and digitize the biopotential detected across the pair of electrodes, and to communicate a patient status on the two-way communication interface.

24. The device of claim 23, wherein the emergency beacon comprises a nonvisible light source.

25. The device of claim 23, wherein the circuitry is further operably configured to enable transmission and activation of the emergency beacon in response to authenticating a command received by the two-way communication interface.
